(19) United States
(12) Patent Application Publication
(10) Pub. No.: US 2017/0049938 A1
(43) Pub. Date: Feb. 23, 2017

(54) METHOD AND MATERIAL FOR COATING ELECTRO-CAUTERY PROBES AND MASKING SURGICAL ODOR

(71) Applicant: Mectra Labs, Inc., Bloomfield, IN (US)

(72) Inventor: Thomas P. Clement, Bloomington, IN (US)

(21) Appl. No.: 15/240,538

(22) Filed: Aug. 18, 2016

Related U.S. Application Data
(60) Provisional application No. 62/206,984, filed on Aug. 19, 2015.

(51) Int. Cl. A61L 31/08; A61L 31/14; C10M 169/04; C09D 7/12; C10M 105/74; A61B 18/14; C09D 5/00

(52) U.S. Cl. CPC A61L 31/08; A61B 18/1482; A61L 31/14; C09D 5/00; C09D 7/1233; C10M 105/74; C10M 169/04; A61B 2018/0013

(57) ABSTRACT

A coating is provided for use in an electro-cautery probe of a cauterization device to mask odors generated during a cauterization procedure. The coating is also provided to increase the longevity of the odor masking and prevent tissue charring.

### CROSS REFERENCE TO RELATED APPLICATIONS

This application claims the benefit of and priority to U.S. Provisional Patent Application No. 62/206,984, filed 19 Aug. 2015, the disclosure of which is now incorporated herein by reference.

### FIELD OF THE DISCLOSURE

The present invention relates to a method and material for coating electro-cautery probes and masking surgical odor.

### BACKGROUND

Surgical processes of various types often utilize electro-cautery devices for cutting and/or cauterizing, some of which are mono-polar devices and others of which are bi-polar devices. These devices are well known for use in surgery and can be acquired from various suppliers. Such electro-cautery probes, which are often referred to as “tips” cause a burning flesh smoke and odor to permeate the surgical theater. The odor is unpleasant and may be distracting to medical personnel.

The prior art has suggested a variety of man-made or synthetic materials for coating electro-cautery probes. Such prior art may reduce charring or size of a smoke plume. Other prior art suggests centralized smoke evacuation systems or various ventilation systems. However, the odor created by electro cautery procedures is unavoidable. Thus, there is a need to mask this odor to create a more tolerable working environment.

Also, in surgery procedures involving inserting instruments into other instruments, there is a need for lubrication of the instruments.

### SUMMARY

The present disclosure comprises one or more of the following features or elements or the combination thereof:

A coating is provided for electro-cautery probes to reduce the charring and to resist sticking of the tissue on the electro-cautery probe and mask the odor of cauterizing tissue. The coating, which is applied to the probes, is effective for masking such odors. The coating is also effective as a lubricant for use in surgery to facilitate movement of instruments relative to each other. For example, the coating may be used to facilitate insertion of instruments into trocars in laparoscopic surgery. Illustratively, the coating is provided in a liquid form.

The illustrative coating includes an amphiphilic lipid and more specifically may include an amphiphilic phospholipid and an odor-masking biocompatible compound. The odor-masking biocompatible compound may include hexyl acetate, fructones, ethyl methylphenylglycide, lactones, or other aroma compounds.

The coating may illustratively be applied to the electro-cautery probes by dipping the probes in or wiping the probes with the coating. For example, a method for coating electro-cautery probes in a surgery procedure in an operating room includes providing a cauterization device having at least one electro-cautery probe to cut and/or cauterize tissue, providing a container of odor-masking liquid coating for masking odor generated at the tip of the at least one electro-cautery probe; and applying the odor-masking liquid coating to the tip of the electro-cautery probe. A lubricating liquid coating may also be provided to be mixed with the odor-masking liquid coating or applied to the tip of the probe separately from the odor-masking liquid coating to prevent skin charring and prevent the odor-masking coating from burning off too quickly. The illustrative cauterization device and all cauterization devices disclosed herein may be bi-polar or mono-polar cauterization devices.

Further, a kit may be provided for lubricating an electro-cautery probe of a cauterization device. The kit may include the above-discussed lubricated coating, a container containing the coating, a plurality of biocompatible scented compounds in individual containers, and a sterile pad having a top surface formed to receive a portion of the coating. The pad may include a foam portion, an adhesive provided on at least one surface of the foam portion, and a removable backing coupled to the adhesive.

In a presently preferred aspect of the invention, the coating includes a lubricious coating and a scented compound and is supplied as a liquid for use by surgeons or technicians directly in the operating room to coat the electro-cautery probes before and during surgery without any heating or curing. Small containers of an odor-masking coating may be supplied with sterile pads for applying the coating to the probes. Further, a surgeon, technician, or other user may attach the pad of the kit to an area adjacent a cauterization site prior to the cauterization procedure and may place a portion of the coating onto a top surface of the pad. The user may then dip the tip of the electro-cautery probe into the coating provided on the top surface of the pad prior to cauterizing a patient's tissue. The user may re-dip the tip of the electro-cautery probe into the coating as often as desired or necessary throughout the cauterization process.

In another embodiment, the lubricious coating and the scented compound may be provided as separate coatings, each supplied with a sterile pad for alternating application of the scented coating and the lubricious coating to the probes. The user may then dip the tip of the electro-cautery probe into the each coating provided on the top surface of each pad prior to cauterizing a patient's tissue. The user may re-dip the tip of the electro-cautery probe into one or both coatings as often as desired or necessary throughout the cauterization process.

The same lubricious and odor-masking, or scented, coating discussed above for coating electro-cautery probes may also be supplied to surgeons or technicians for use in coating the blades of bipolar scissors, for example, as well as coating surgical instruments or portions of instruments to mask odors generated in surgical applications.

In another embodiment, a coating for use in lubricating an electro-cautery probe of a cauterization device to resist sticking of the tissue on the electro-cautery probe and mask the odor of cauterizing tissue may comprise an amphiphilic lipid and an odor-masking compound. In some embodiments, the odor-masking compound is a scented compound.

In some embodiments, the scented compound is biocompatible. In some embodiments, the scented compound is selected from the group consisting of hexyl acetate, fructone, ethyl methylphenylglycidate, and combinations thereof. In some embodiments, the scented compound is hexyl acetate. In some embodiments, the scented compound is fructone. In some embodiments, the scented compound is ethyl methylphenylglycidate. In some embodiments, the scented compound comprises a lactone.

According to another aspect of the present disclosure, a kit for masking a cauterizing odor generated by an electro-cautery probe may include a coating, a plurality of containers of biocompatible scented compounds, a container containing the coating and with headspace to receive one of the biocompatible scented compounds, and a pad having a top surface formed to receive a portion of the coating. The coating may comprise an amphiphilic lipid.

In some embodiments, the coating, the compounds, the container, and the pad are sterile. In some embodiments, each of the plurality of containers of biocompatible scented compounds contains a different scent.

In some embodiments, the kit may further comprise a cauterization device having at least one electro-cautery probe.

According to another aspect of the present disclosure, an electro-cautery probe for attachment to and use with a cauterization device to cut and/or cauterize tissue of a patient in surgery may comprise at least one surface of the electro-cautery probe which contacts a patient's tissue to be cut and/or cauterized and a coating having an odor-masking compound on the at least one surface.

In some embodiments, the odor-masking compound is a biocompatible scented compound. In some embodiments, the coating further comprises lecithin. In some embodiments, the coating cooperates with heat generated at the at least one surface of the electro-cautery probe during cauterization or cutting to increase the intensity of a scent of an odor-masking compound.

According to another aspect of the present disclosure, an electro-cautery probe for attachment to and use with a cauterization device to cut and/or cauterize tissue of a patient in surgery, the electro-cautery probe may have at least a surface of the electro-cautery probe which contacts a patient's tissue to be cut and/or cauterized coated with a coating comprising lecithin and means for masking odor generated during cutting or cauterizing the patient's tissue.

In some embodiments, the means for masking odor generated during cutting or cauterizing comprises a scented compound coupled to a cauterizing tip of the probe. In some embodiments, the energy generated during the cauterization increases the scent throw of the scented compound.

According to another aspect of the present disclosure, a method of coating electro-cautery probes in an operating room may comprise providing a cauterization device having at least one electro-cautery probe to cut and/or cauterize tissue, providing a container of odor-masking liquid coating for masking odor generated at the tip of the at least one electro-cautery probe, and applying the odor-masking liquid coating to the tip of the electro-cautery probe.

In some embodiments, the method may further comprise providing a container of lubricating liquid coating for lubricating the at least one electro-cautery probe and applying the lubricating liquid coating in the container to the tip of the electro-cautery probe. In some embodiments, the method may further comprise providing a container of lubricating liquid coating and combining the odor-masking liquid into the lubricating liquid coatings. In some embodiments, the step of applying the odor-masking liquid coating comprises applying the combined odor-masking and lubricating liquid coatings.

In some embodiments, the container is a spray bottle and applying the liquid coating includes spraying the coating onto the at least one electro-cautery probe. In some embodiments, the spray bottle is a pump-driven spray bottle.

According to another aspect of the invention, a method of coating an electro-cautery probe of a cauterization device with a coating may comprise attaching a first pad having a top surface to receive a portion of a first coating to an area adjacent a cauterization site prior to the cauterization procedure. In some embodiments, the method may further comprise dipping a tip of the electro-cautery probe into the first coating provided on the top surface of the first pad prior to cauterizing a patient's tissue. In some embodiments, the method may further comprise attaching a second pad having a top surface to receive a portion of a second coating to an area adjacent a cauterization site prior to the cauterization procedure. In some embodiments, the method may further comprise dipping a tip of the electro-cautery probe into the second coating provided on the top surface of the second pad prior to cauterizing a patient's tissue. In some embodiments, the dipping steps are repeated during the cauterization procedure as often as necessary.

Features of the present disclosure will become apparent to those skilled in the art upon consideration of the following detailed description of illustrative embodiments exemplifying the best mode of carrying out the disclosure as presently perceived.

### BRIEF DESCRIPTION OF THE DRAWINGS

The detailed description particularly refers to the accompanying figures in which:

FIG. 1 is a perspective view of a probe-coating kit showing two foam blocks or pads, a container containing a lubricating liquid coating, a plurality of containers each containing a biocompatible scented compound, and an instruction pamphlet and also showing a cauterization device and a surgical drape for use during various surgical procedures such as cauterization, for example;

FIG. 2 is a perspective view of the components of the kit and cauterization device in use during surgery showing a first foam block adhered to the drape and showing the lubricating liquid solution being poured onto the first foam block, and further showing an odor-masking liquid being poured on a second foam block adhered to the drape such that a tip of an electro-cautery probe of the cauterization device may be dipped as needed into the odor-masking liquid and/or lubricating liquid solution during the cauterization process in order to substantially prevent tissue fragments from collecting on the tip of the electro-cautery probe;

FIG. 3 is a perspective view showing the tip of the electro-cautery probe being dipped into a solution of lubricating liquid solution and a an odor-masking liquid which was poured onto the foam block; and

FIG. 3A is a perspective view showing the tip of the electro-cautery probe coated with a non-stick, odor-masking liquid coating comprising the lubricating liquid solution and the odor-masking liquid solution.

### DETAILED DESCRIPTION OF THE DRAWINGS

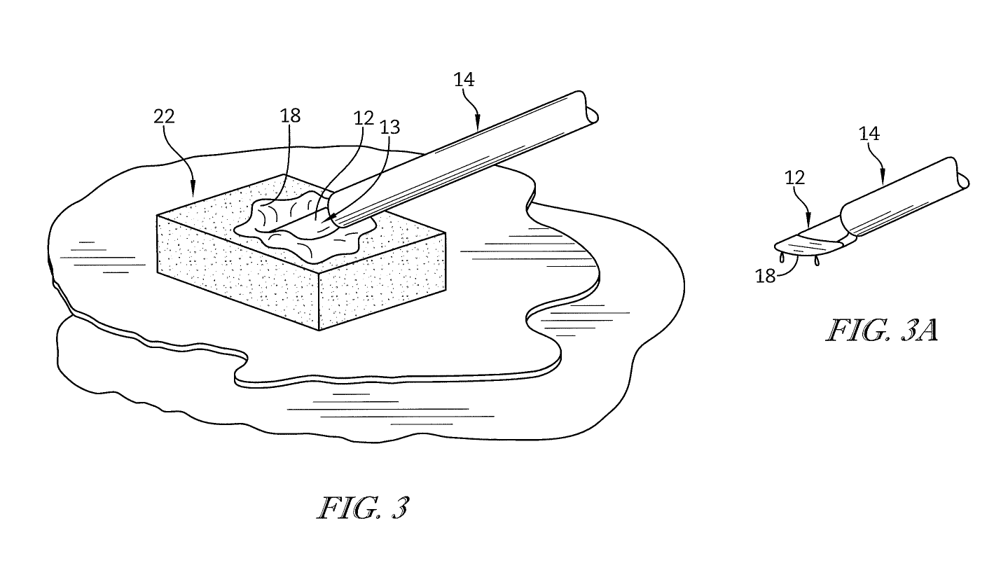

A non-stick, odor-masking liquid coating 18 is provided for coating a tip 12 of an electro-cautery probe 13 of an illustrative cauterization device 14 in order to prevent tissue fragments from sticking to and coating the tip 12 of the cauterization device 14 and to mask the burning flesh odor generated during a cut and/or cauterization procedure, such as that shown in FIG. 3, for example. The term cauterization procedure herein refers to any type of surgical cut and/or cauterization-type procedure. Illustratively, the non-stick, odor-masking coating 18 is effective for preventing such sticking or accumulation of tissue and for masking odor, and may also be effective as a lubricant for use in other surgeries to facilitate movement of instruments relative to each other. For example, the non-stick odor-masking coating 18 may be used to facilitate insertion of instruments into trocars in laparoscopic surgery.

The illustrative coating 18 is made from a natural or biological material which is safe to use during surgery. The illustrative coating 18 is a combination of a scented coating 8, sometimes called an odor-masking liquid coating 8, and a lubricating coating 10, sometimes called a lubricating liquid coating 10 or lubricious coating 10. In one embodiment, the scented coating 8 is a liquid including a naturally occurring biocompatible aroma compound.

In some embodiments, the scented coating 8 comprises a scented compound, sometimes called a biocompatible scented compound. The term biocompatible, as used herein, refers to a material that does not elicit a substantial detrimental response in the host or patient. Illustratively, the scented compound may sometimes be called a biocompatible aroma compound. Illustratively, the scented compounds can be alkyl esters, aryl esters, terpenes, hydroxy terpenes, cyclic terpenes, lactones, alkyl aldehydes, aryl aldehydes, combinations thereof, and any suitable alternative.

Non-limiting examples of scented compounds include the alkyl esters hexyl acetate, fructone, ethyl methylphenylglycidate, combinations thereof, and any suitable alternative. Without being bound by theory, it is postulated that hexyl acetate and or fructone may be a scented compound used to provide a fruity odor. In some embodiments, ethyl methylphenylglycidate may provide a strawberry scent.

Non-limiting examples of lactones include gamma-decalactone, gamma-nonalactone, massoia lactone, combinations thereof, and any suitable alternative. In some embodiments, lactones may be provided for a coconut/peach scent. Other biocompatible aroma compounds and/or combinations of compounds may be provided. The scented compounds may be provided in solution to provide an easy coating medium and ease of combination with other coatings.

The lubricating coating 10 may be any number of known coatings provided in the art. Specific examples of a lubricating coating, such as lecithin, are provided in U.S. Pat. No. 7,217,270 which is hereby incorporated by reference. Illustratively, the lubricating coating 10 will minimize the burning of the scented coating 8 and slow the evaporation of the scent during a cauterization procedure.

In some embodiments, the lubricating coating 10 comprises an amphiphilic lipid. As used herein, the term “amphiphilic” refers to a property where a molecule has both a polar portion and a non-polar portion. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt.

Without being bound by theory, it is postulated that amphiphilic lipids are particularly suited for use as coatings of electro-cautery probes due to their stabilizing nature. Amphiphilic lipids are able to stabilize both positive and negative sites contained or located on the metallic surface or tip, such as tip 12, of the electro-cautery probe 13 of a cauterization device, such as device 14, for example.

Lecithin is an example of an amphiphilic lipid and as such, forms a phospholipid bilayer where the hydrophilic (polar) heads face their surroundings, which are oftentimes aqueous, and the hydrophobic tails face each other. This structure allows lecithin to acts as an emulsifier and is, at least in part, what provides lecithin with the non-stick property. Although lecithin is disclosed herein, it is within the scope of this disclosure for lubricating coating 10 to include any amphiphilic lipid found to be biocompatible or safe and non-toxic to permit use on patients with cauterization devices or other surgical instruments.

As used herein, the term lecithin includes any phosphatidylcholine derivatives of glycerol, and having the general structure depicted by Formula I:

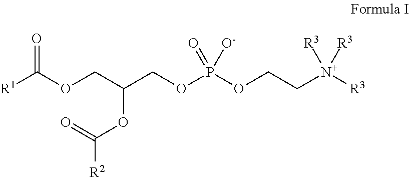

wherein R¹ and R² are each independently selected from alkyl, alkenyl, arylalkyl, and arylalkenyl, each of which may be optionally substituted, and including C₁-C₃₀ alkyl and C₁-C₃₀ alkenyl, such as but not limited to stearic, palmitic, oleic, palmitoleic, linoleic, linolenic, and arachidonic acid side chains; and R³ is in each instance independently selected from C₁-C₄ alkyl, including methyl and ethyl.

It is appreciated that the chiral center in Formula I may be of either stereo configuration, or alternatively, a mixture of stereoisomers may be present. Such a mixture of stereoisomers may have an equal population of each stereoisomer, as in a racemic mixture, or may have an unequal population of each stereoisomer, and thus exhibit optical activity.

It is also appreciated that coating components for use as lubricants for electro-cautery probes may be advantageously selected from those components that are stable to higher temperatures, such as those higher temperatures which may be observed on the surface of electro-cautery probes when in use, for example.

Formulations of the coating compositions described herein may also include one or more other components such as alcohols, fatty acids, oils, surfactants, water, dispersing agents, and thixotropic agents. Dispersing agents include, but are not limited to, propylene glycol based ethers, propylene glycol based ether acetates, ethylene glycol based ethers, ethylene glycol based ether acetates, and mixtures thereof.

Illustratively, as mentioned above, the coating 18 is provided to coat the tip 12 of the electro-cautery probe or probes 13 of cauterization device 14. The cauterization device 14 may include bi-polar electro-cautery probes or mono-polar electro-cautery probes. The coating 18 may be used to coat the tips 12 of either bi-polar or mono-polar probes.

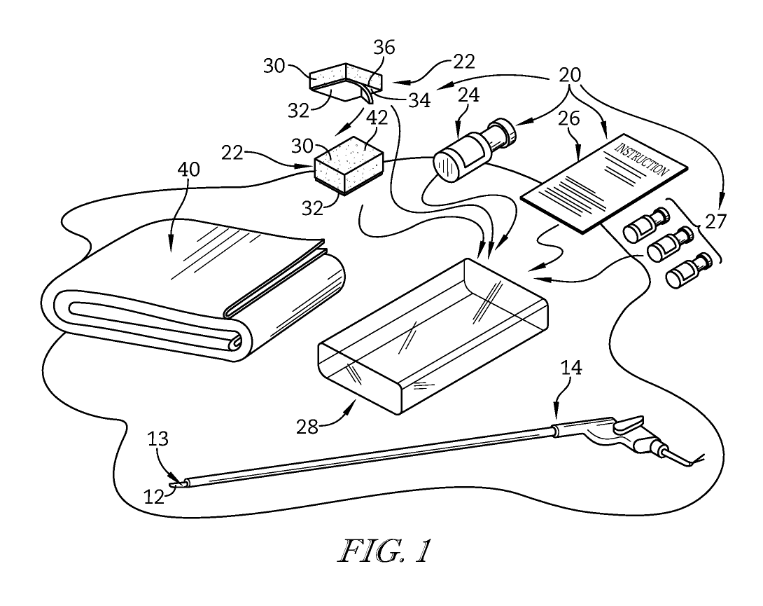

Looking now to FIG. 1, a probe-coating kit 20 is provided. In the illustrative embodiment, the kit includes two foam blocks or pads 22, a bottle or container 24 including the lubricating liquid coating 10, and a plurality of scent containers 27, sometimes called scent bottles 27, each containing a biocompatible scented compound as discussed above. The bottle 24 may be provided with headspace sufficient to allow liquid from one or more of the plurality of scent containers 27 to be combined therein. Although the exemplary embodiment shows three scent containers 27, any number of scent containers 27 may be provided to provide more or fewer options for customizable user scent preferences. Although foam blocks or pads 22 are made of foam, it is within the scope of the disclosure for the pads 22 to be made of other suitable materials such as cotton, for example. Some kits may include the tool to be coated such as a cauterization device 14 and probe 13.

Illustratively, an instruction sheet 26 providing instructions for use of the foam blocks 22 and lubricating coating 10, as described below, is also provided. The foam blocks 22, bottle 24, and scent container 27 and instruction sheet 26 are placed within a clear, plastic pouch 28, sealed, and sterilized prior to use by a surgeon or technician, for example, during surgery or other cauterization process. It is also within this disclosure for foam blocks 22, bottle 24, scent container 27, and instruction sheet 26 to be carried within another suitable container which may be sterilized prior to use by a surgeon or technician.

Each illustrative foam block 22 has a surface area of approximately two inches by two inches and includes a foam portion 30, a removable backing 32, and an adhesive 34 provided on a bottom surface 36 of the foam portion 30. When the foam block 22 is not in use, the removable backing 32 is coupled to the bottom surface 36 of the foam square in order to cover the adhesive 34.

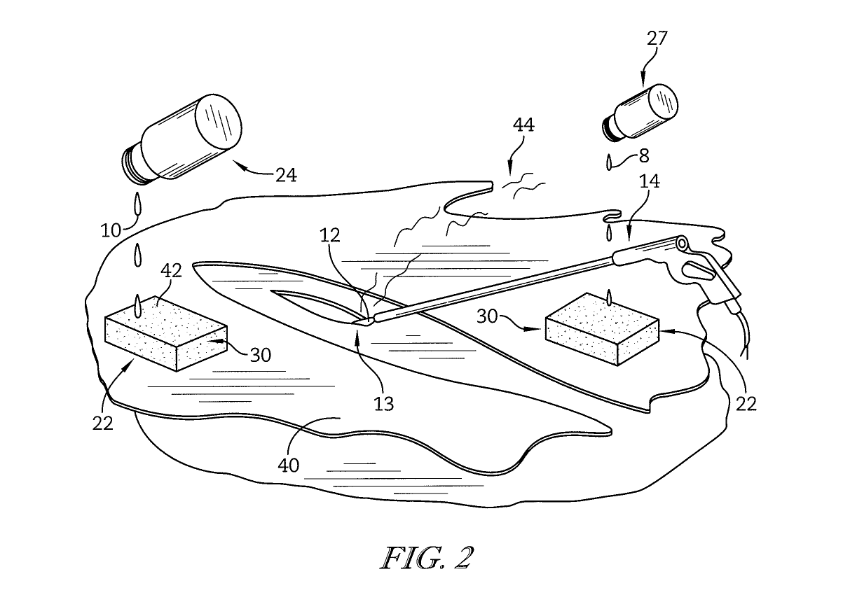

In using kit 20, a doctor, technician, or other user opens the plastic pouch 28 and removes the contents (foam blocks 22, bottle 24 containing lubricating coating 10, scent container 27 containing scented coatings 8, and instruction sheet 26). Backing 32 of one or both of the foam blocks 22 is then removed to expose the adhesive 34 thereon, as shown in FIG. 1, for example. By placing the adhesive-side or bottom surface 36 down, foam portion 30 of foam block 22 may then be coupled onto an outer surface of a patient drape 40, for example, such as that shown in FIG. 2. Foam portion 30 may be adhered to other convenient surfaces near the location where the cauterization is to take place as well, for example. A top surface 42 of foam block 22 provides a surface for placing and holding a desired amount of coating 10, 8. Although foam blocks 22 are provided with kit 20, it is within the scope of this disclosure to include other devices for providing a suitable portable, sterile surface upon which a portion of coating 10, 8 may be conveniently held during the cauterization process such as a small tray, for example.

Thus, once one or both of the foam blocks 22 have been secured to drape 40 or another nearby area, the user opens the bottle 24 containing lubricious coating 10 and pours a desired amount of lubricious coating 10 onto top surface 42 of foam block 22, as shown in FIG. 2 in order to provide a readily-accessible amount of lubricious coating 10 within the vicinity of the area in which the cauterization device 14 is to be used. A desired amount of scented coating 8 is poured onto another foam block 22 to provide a readily accessible amount of scented coating 8 within the vicinity of the area in which the cauterization device 14 is to be used. As mentioned above, another device or apparatus may be used other than foam blocks 22 to conveniently contain or hold a portion of lubricious coating 10 and a portion of scented coating 8 thereon. Further, the device used may be absorbent, such as foam block 22, to contain and hold the lubricating coating 10 and scented coating 8. The surface-providing device may also be made of a material which is not absorbent as well.

The tip 12, of the electro-cautery probe 13 of cauterization device 14 can be dipped in the scented coating 8 located on foam block 22 followed by the lubricious coating 10 located on a second foam block 22. It is contemplated that in preferred embodiments, the tip is first dipped in the scented coating 8 followed by the lubricious coating 10 on each pad 22. However, the tip 12 may be dipped in the opposite order, or may be dipped multiple times alternating in each coating 10, 8, to create a layering effect. If additional coatings 10, 8 are needed during the procedure, the user may pour additional coatings 10, 8 onto their respective foam squares 22. This prevents the user from having to dip the tip 12 directly into the containers 24, 27, so that the remaining coating in the containers may be used in future procedures. As seen in FIG. 2, the heat generated from the cauterization procedure will carry the scent 44, or increase the scent throw, of the scented coating 8. The heat, typically carrying the odor of the burning flesh from the procedure point will be masked by the scent coating 8 provided at the point of cauterization.

As shown in FIGS. 3 and 3A, an alternative means or method is provided to coat the tip 12 of the probe 13. In this embodiment, coating 18 is a mixture of lubricious coating 10 and scented coating 8. The mixture may be formed by pouring the scented coating 8 into a headspace area left in the container 24 containing the lubricious coating 10. Alternatively, the coating 18 may be provided to user premixed in a container (not shown). In this manner, a single foam square is required 22 to hold the coating 18. Tip 12 only requires dipping on a single foam block 22 to coat the tip 12 to obtain both lubricious and odor-masking qualities. The user may re-coat tip 12 as often as necessary by dipping tip 12 into coating 18 provided on foam block 22. If additional coating 18 is needed during the procedure, the user may pour additional coating 18 onto the foam block 22. This allows the coating 18 which remains within the bottle to remain sterile and usable for future procedures. The surgeon or technician may also use the pad 22 to wipe coating 18 onto tip 12 in addition to using the pad 22, as discussed above, to hold or contain coating 18 for dipping of the tip 12 therein.

Therefore, a means or method of coating an electro-cautery probe tip 12 is provided. This method includes dipping or wiping the electro-cautery probe tip 12 into coating 18. More specifically, this method includes placing or adhering one or more portable, sterile surfaces, such as surface 42 of foam blocks 22, onto surgical drape 40, or another convenient area, pouring a selected scented coating 8 into a lubricious coating 10 in container 24 and mixing them, and dispensing a desired amount of coating 18 onto the top surface 42. The mixing can be achieved by reapplying the cap to container 24 and shaking vigorously. The method next includes dipping the electrode tip 12 onto the foam surface containing coating 18. The dipping steps may be repeated as often as desired or necessary.

As mentioned above, instruction sheet 26 is also provided in kit 20 and contained within plastic pouch 28. Instruction sheet 26 includes instructions to inform the surgeon or technician how to properly use the contents of kit 20 to coat the tip 12 of cauterization device 14 with the lubricious coating 10 contained within bottle or container 24 and the scented coating 8 contained in scent container 27. Illustratively, the instructions on the instruction sheet 26 may read as follows:
1. Place contents of pouch onto sterile field.
2. Remove backing from foam and stick adhesive side down onto patient drape.
3. Select a scent and remove cap from scent bottle pour into lubricious coating bottle, shake to combine
4. Dispense desired amount of lubricious coating onto foam.
5. Wipe electrode tip onto foam containing coating.
6. Repeat as frequently as desired.

Additional or alternative instructions on the instructions sheet 26 may read as follows:
1. Place contents of pouch onto sterile field.
2. Remove backing from two foam pads and stick adhesive side down onto patient drape.
3. Select a scent and dispense a desired amount of scent coating onto a foam pad.
4. Pour a desired amount of lubricious coating onto the other foam pad.
5. Wipe electrode tip on foam containing scent coating.
6. Wipe electrode tip on foam containing lubricious coating.
7. Repeat as frequently as desired.

Although coating 18 is described above for use with cauterization devices 14 having electro-cautery probes 13, it is understood that the term cauterization device includes other suitable devices used in cauterization-type processes, such as bipolar scissors (not shown), for example, in order to prevent charring or accumulation of tissue onto the device. Illustrative non-stick odor-masking coating 18 may be used to coat the scissor blades (not shown) of a bipolar scissors in the manner discussed above with respect to devices 14. Other surgical devices may also be or coated with coating 18. Furthermore, various coating dispensers may be provided on the probe such as syringe-like mechanism attached to the probe so that the tip may be coated during cauterization.

Although the embodiments all describe scented coating 8 being used in layered or mixed combination with lubricious coating 10, it is contemplated that scented coating 8 may be used by itself or with other coatings. Coating 18 may be used to lubricate various surgical instruments to facilitate movement of the instruments relative to each other during use. For example, coating 18 may be used to facilitate insertion of instruments into trocars in laparoscopic surgery.

While the disclosure has been illustrated and described in detail in the foregoing drawings and description, the same is to be considered as exemplary and not restrictive in character, it being understood that only illustrative embodiments thereof have been shown and described and that all changes and modifications that come within the spirit of the disclosure are desired to be protected.

1. A coating for lubricating an electro-cautery probe of a cauterization device to resist sticking of tissue on the electro-cautery probe and mask the odor of cauterizing tissue, the coating comprising:
an amphiphilic lipid; and
an odor-masking compound;
wherein the odor-masking compound is a scented compound.

2. The coating of claim 1, wherein the scented compound is biocompatible.

3. The coating of claim 2, wherein the scented compound is selected from the group consisting of hexyl acetate, fructone, ethyl methylphenylglycidate, and combinations thereof.

4. The coating of claim 1, wherein the scented compound comprises a lactone.

5. The coating of claim 1, wherein the amphiphilic lipid comprises lecithin.

6. A kit for masking a cauterizing odor generated by an electro-cautery probe comprising:
a lubricating coating comprising an amphiphilic lipid;
a plurality of scent containers, each containing a biocompatible scented compound;
a container containing the lubricating coating and having headspace to receive a biocompatible scented compound; and
a pad having a top surface formed to receive a portion of the coating.

7. The kit of claim 6, wherein the lubricating coating, the biocompatible scented compounds, the containers, and the pad are sterile.

8. The kit of claim 6, wherein each container of the plurality of containers of biocompatible scented compounds contain a different biocompatible scented compound.

9. The kit of claim 6, further comprising a cauterization device having at least one electro-cautery probe.

10. An electro-cautery probe for attachment to and use with a cauterization device to cut and/or cauterize tissue of a patient in surgery, the electro-cautery probe comprising:
at least one surface configured to contact a patient's tissue to be cut and/or cauterized; and
a coating comprising an odor-masking compound on the at least one surface.

11. The electro-cautery probe of claim 10, wherein the odor-masking compound is a biocompatible scented compound selected from the group consisting of hexyl acetate, fructone, ethyl methylphenylglycidate, a lactone, and combinations thereof.

12. The electro-cautery probe of claim 10, wherein the coating further comprises lecithin.

13. A method of coating an electro-cautery probe in an operating room comprising:
providing a cauterization device having at least one electro-cautery probe;
providing a container of an odor-masking liquid coating for masking odor generated at the tip of the at least one electro-cautery probe; and
applying the odor-masking liquid coating to the tip of the electro-cautery probe.

14. The method of claim 13, further comprising providing a container of lubricating liquid coating for lubricating the at least one electro-cautery probe and applying the lubricating liquid coating in the container to the tip of the electro-cautery probe.

15. The method of claim 13, further comprising providing a container of lubricating liquid coating; and combining the odor-masking liquid coating and the lubricating liquid coating; wherein applying the odor-masking liquid coating comprises applying the combined odor-masking liquid coating and the lubricating liquid coating.

16. The method of claim 13, wherein the container is a spray bottle and applying the odor-masking liquid coating includes spraying the odor-masking liquid coating onto the at least one electro-cautery probe.

17. The method of claim 16, wherein the spray bottle is a pump-driven spray bottle.

18. The method of claim 13, further comprising:
attaching a first pad having a top surface to receive a portion of a first coating to an area adjacent a cauterization site prior to the cauterization procedure,
dipping a tip of the electro-cautery probe into the first coating provided on the top surface of the first pad prior to cauterizing a patient's tissue;
attaching a second pad having a top surface to receive a portion of a second coating to an area adjacent the cauterization site prior to the cauterization procedure,
dipping a tip of the electro-cautery probe into the second coating provided on the top surface of the second pad prior to cauterizing the patient's tissue.

19. The method of claim 18, wherein the dipping steps are repeated during the cauterization procedure as often as necessary.

20. The method of claim 18, wherein the first coating comprises the lubricating liquid coating and the second coating comprises the odor-masking liquid coating.
